(19) United States
(12) Patent Application Publication
(10) Pub. No.: US 2021/0389331 A1
(43) Pub. Date: Dec. 16, 2021

(54) Method for Diagnosing Intermittent Claudication and Chronic Limb-Threatening Ischemia

(71) Applicant: McMaster University, Hamilton (CA)

(72) Inventor: Phillip Britz-McKibbin, Hamilton (CA)

(21) Appl. No.: 17/333,189

(22) Filed: May 28, 2021

Related U.S. Application Data
(60) Provisional application No. 63/031,740, filed on May 29, 2020.

(51) Int. Cl. G01N 33/68

(52) U.S. Cl. CPC G01N 33/6893

(57) ABSTRACT

A method of diagnosing peripheral artery disease in a mammalian subject is provided, including a method of distinguishing advanced disease from a less advanced form, e.g. CLTI from IC, based on the level of one or more metabolic biomarkers selected from: creatine, creatinine, carnitine, propionylcarnitine, cystine, lysine, tyrosine, histidine, phenylacetylglutamine, oxoproline, arginine, monomethylarginine, a fatty acid biomarker selected from the group consisting of stearic acid, linoleic acid, heptadecanoic acid, palmitic acid, oleic acid, heptadecenoic acid, pentadecanoic acid and eicosadienoic, and a ratiometric biomarker comprising at least one of the metabolic biomarkers.

### FIELD

The present application relates to the field of peripheral artery disease (PAD) and in particular, relates to methods of diagnosing PAD and differentiating between different stages of PAD, such as intermittent claudication (IC) and chronic limb-threatening ischemia (CLTI).

### BACKGROUND

Peripheral artery disease (PAD) is a form of atherosclerosis manifested in the lower extremities leading to a cascade of symptoms from insufficient blood flow, including impaired/painful walking, reduced functional capacity, ischemic myopathy, and recurrent skin lesions [1]. PAD is also associated with higher risk for cardiovascular events such as myocardial infarction, stroke, and vascular death [2]. Although most PAD patients are asymptomatic, patients with PAD usually present with intermittent claudication (IC), characterized with varying degrees of pain in leg muscles induced by walking. If left untreated, patients can progress to a severe end-stage form of PAD known as chronic limb-threatening ischemia (CLTI), which is characterized by rest pain, non-healing ischemic ulcers, and gangrene requiring limb amputation [2]. Disease progression in PAD is highly variable and unpredictable as some CLTI patients who undergo amputations do not exhibit any PAD symptoms 6 months before onset [3]. In fact, cardiovascular events are more prevalent among CLTI patients than coronary artery disease (CAD) indicating significant associated morbidity [3]. Although CLTI accounts for less than 5% of total PAD diagnosis, survivorship is poor with a 5-year mortality rate of about 50% [4]. As a result, there is urgent need for understanding the mechanisms of PAD progression for early detection of CLTI that also guides evidence-based treatment decisions [5].

Despite a high estimated prevalence of 10-20% among older persons, PAD is often undiagnosed and/or untreated in the primary care setting with most physicians unaware of the diagnosis [6]. In practice, diagnosis is confirmed in symptomatic patients by an abnormal resting ankle-brachial index (ABI) below 0.9, which is determined by the ratio of the systolic blood pressure at the ankle of the affected leg to the upper arm using a Doppler ultrasound blood flow detector [1]. Usually, ABI is a reliable diagnostic tool for PAD diagnosis that may also predict atherosclerotic PAD mortality when performed in accredited laboratories with specialized equipment and training [7]. However, in 25% of diabetic patients, ABI lacks sensitivity to diagnose patients with PAD due to peripheral arterial stiffening from calcification [8]. Also, the benefit of routine screening of PAD risk in asymptomatic patients using ABI remains inconclusive based on recent findings from the US Preventive Services Task Force [9]. For these reasons, specific and sensitive blood-based biomarkers are needed for PAD diagnosis and risk assessment that is applicable for routine testing in a clinical setting, including surveillance of high-risk CLTI patients following revascularization interventions.

Metabolomics offers a systemic approach for molecular phenotyping of complex biological processes underlying cardiovascular diseases (CVD) for new advances in precision medicine and drug development [10]. Comprehensive metabolite profiling using high field nuclear magnetic resonance (NMR) and increasingly high resolution mass spectrometry (MS) enables the discovery of clinically relevant biomarkers associated with atherosclerosis that reflect dynamic interactions between host, gut microbiota, and dietary exposures, such as trimethylamine-N-oxide [11]. Growing evidence also demonstrates that elevated plasma branched-chain amino acid concentrations increase the risk for stroke that may be counteracted by prudent diet modifications [12]. However, most metabolomic studies to date have focused on identifying aberrant metabolic pathways in CAD as compared to standard predictors [13]. In contrast, there have been few reports using metabolomics to understand the pathophysiology of PAD, which is prone to confounding since older patients often suffer from other comorbidities, including type 2 diabetes, chronic kidney disease and/or cardiovascular events [14-17].

### SUMMARY

Distinct metabolic phenotypes of PAD patients have now been determined, thereby permitting diagnosis of PAD, as well as differentiation between the stages of PAD, namely intermittent claudication (IC) and the more severe form of PAD, chronic limb-threatening ischemia (CLTI). This differential diagnosis allows for improved patient risk stratification, therapeutic monitoring, as well as prognosis of PAD patients who may progress to CLTI. Metabolic biomarkers for early detection of PAD offer a more reliable approach for routine clinical testing than conventional ABI measurements, as well as clinical presentations that suffer from poor sensitivity.

In one aspect of the invention, a method of diagnosing peripheral artery disease (PAD) in a mammalian subject is provided comprising:

i) detecting in a biological sample from the subject the level of one or more metabolic biomarkers selected from the group consisting of: creatine, creatinine, carnitine, propionylcarnitine, cystine, lysine, tyrosine, histidine, phenylacetylglutamine, oxoproline, arginine, monomethylarginine, a fatty acid biomarker selected from the group consisting of stearic acid, linoleic acid, heptadecanoic acid, palmitic acid, oleic acid, heptadecenoic acid, pentadecanoic acid and eicosadienoic acid, and a ratiometric biomarker comprising at least one of the metabolic biomarkers;

ii) comparing the level of the one or more detected biomarkers to the corresponding level in a non-PAD control; and

iii) determining that the subject has PAD when the level of the one or more detected biomarkers is statistically different from the corresponding level in the non-PAD control.

In another aspect, a method of distinguishing chronic limb-threatening ischemia (CLTI) from intermittent claudication (IC) in a mammalian subject is provided comprising:

i) detecting in a biological sample from the subject the level of one or more metabolic biomarkers selected from the group consisting of: creatine, creatinine, carnitine, propionylcarnitine, cystine, lysine, tyrosine, histidine, phenylacetylglutamine, oxoproline, arginine, monomethylarginine, a fatty acid biomarker selected from the group consisting of stearic acid, linoleic acid, heptadecanoic acid, palmitic acid, oleic acid, heptadecenoic acid, pentadecanoic acid and eicosadienoic acid, and a ratiometric biomarker comprising at least one of the metabolic biomarkers;

ii) comparing the level of the one or more detected biomarkers to the corresponding level in an IC control; and

iii) determining that the subject has CLTI when the level of the one or more detected biomarkers is statistically different from the corresponding level in the IC control.

In an embodiment, the present methods may be used to predict disease progression and/or monitor treatment responses by monitoring the levels of selected biomarkers over time.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### BRIEF DESCRIPTION OF THE DRAWINGS

The embodiments of the application are described in greater detail with reference to the attached drawings in which:

FIG. 1 shows serum metabolites identified by multiplexed separations in exemplary embodiments of the application. (A) Multiplexed separations of extracts of serum samples of PAD patients and non-PAD controls for comprehensive metabolomics analysis using multisegment injection-capillary electrophoresis-mass spectrometry (MSI-(NA)CE-MS) under 3 modes of detection, where the black trace depicts a total electropherogram under (aqueous) positive mode. This method relies on a serial injection of six randomized samples and a QC within each run to enhance sample throughput as shown for (B) carnitine as an example of the rigorous data filtering process for metabolite authentication from spurious signals by confirming precision of six replicate injections with no background signal in blank (0) injection followed by confirmation by its high resolution MS spectrum and spike/recovery study in pooled serum. (C) Various serial injection configurations are illustrated for stearic acid (18:0) determination when analyzing seven independent samples within a single run by MSI-NACE-MS including replicate injections of pooled serum as QC with blank extract to filter out spurious signals and assess technical precision and lack of sample carry-over, randomized analysis of six serum extract samples from individual PAD patients together with a QC normalized to the deuterated internal standard, and 7-point calibration curve for reliable quantification of stearic acid.

FIG. 2 shows the precision of the MSE-CE-MS method in exemplary embodiments of the application. Left: 2D scores plot from PCA of glog-transformed and autoscaled serum metabolome data used to compare the inter-subject biological variance of the serum metabolome relative to the technical variance from repeat analysis of pooled serum QC samples for (A) the hydrophilic serum metabolome and (B) the lipophilic serum metabolome. Right: Control charts for (C) the recovery standard (F-Phe) measured under aqueous positive ion mode, for (D) the recovery standard NMS measured under aqueous negative ion mode, and for (E) the recovery standard 14-d27 measured under non-aqueous negative mode for all serum and QC samples demonstrate acceptable precision (CV=5.2-15.5%) with no outliers exceeding warning limits (±3 s). (F) Bland-Altman percent difference plot for comparing the mutual agreement between serum creatinine concentrations measured independently by MSI-CE-MS and Jaffe methods at two different laboratories from 56 subjects. Overall, the data is randomly distributed with a negligible negative bias of 5.6% with four outliers outside agreement limits. (G) A Passing-Bablok regression analysis demonstrates no significant deviation (dotted lines; 95% confidence interval) from the line of equality (p>0.05) with a slope of 1.17 (black line; regression line).

FIG. 3 shows the differentiation of serum metabolites in CON, IC and CLTI sub-groups in exemplary embodiments of the application. (A) 2D heat map of serum metabolome of PAD patient sub-groups (IC, CLTI) and non-PAD controls (CON) that summarizes overall data structure of this study. (B) A 2D scores plots using PLS-DA model to differentiate the metabolic phenotype of late-stage CLTI (n=18) from early onset IC (n=20) cases as compared to age and sex-matched CON (n=20) based on 85 serum metabolites/lipids (C) Ten top-ranked serum metabolites that differentiate PAD patients and non-PAD controls based on a variable importance in projection (VIP scores>1.5). (D) Correlation matrix depicts two main clusters of circulating metabolites associated with PAD, including circulating amino acids/amines strongly correlated (r˜0.70) to lysine (histidine, tyrosine, monomethylarginine, creatine), as well as long-chain fatty acids (18:2, 20:2, 24:0, 24:1) unlike serum phenyl acetylglutamine.

FIG. 4 shows box-whisker plots illustrating differences in twelve top-ranked metabolites' serum concentrations as compared between chronic limb-threatening ischemia (CLTI) patients (n=18), matched intermittent claudication (IC) patients (n=20) and non-PAD controls (n=20). A one-way ANOVA test was performed to compare means and identify significant changes in circulating metabolite concentrations between the three groups as summarized in Table 2 where a polynomial contrasts analysis depicts most metabolites having a significant linear trend proportional with disease progression. Planned contrasts were conducted by comparing non-PAD controls to PAD cases [IC+CLTI] (contrast 1; long bracket) followed by comparing IC to CLTI (contrast 2; short bracket) reflecting disease status and progression, respectively where test significance is denoted as * p<0.05 and ** p<0.01. Serum stearic acid and carnitine were significantly different between CLTI and IC when using an unpaired t-test for comparison with FDR adjustment (q<0.05).

FIG. 5 shows receiver operating characteristic (ROC) curves and their corresponding box-whisker plots for two top-ranked serum biomarker ratios used for discriminating chronic limb-threatening ischemia (CLTI, n=18) from intermittent claudication (IC, n=20) patients, including (A) stearic acid:carnitine and (B) arginine:propionylcarnitine. Ratiometric ROC curves depict the area under the curve (AUC) and their 95% confidence intervals (blue shaded area). Lower panels depict linear relationship of serum biomarkers of PAD disease progression as a function of abnormal ankle brachial index (ABI<0.90) measurements with moderate Pearson correlation coefficients (r>0.50; p<0.002).

### DETAILED DESCRIPTION

A method of diagnosing PAD in a mammalian subject is provided comprising: i) detecting in a biological sample from the subject the level of one or more metabolic biomarkers selected from the group consisting of: creatine, creatinine, carnitine, propionylcarnitine, cystine, lysine, tyrosine, histidine, phenylacetylglutamine, oxoproline, arginine, monomethylarginine, a fatty acid biomarker selected from the group consisting of stearic acid, linoleic acid, heptadecanoic acid, palmitic acid, oleic acid, heptadecenoic acid, pentadecanoic acid and eicosadienoic acid, and a ratiometric biomarker comprising at least one of the metabolic biomarkers; ii) comparing the level of one or more detected biomarkers to the corresponding level in a non-PAD control; and iii) determining that the subject has PAD when the level of one or more detected biomarkers is statistically different to the corresponding level in the non-PAD control.

To conduct the method, a biological sample is obtained from a mammalian subject, e.g. a human subject. The term “biological sample” is meant to encompass any human sample that may contain relevant metabolites, including biological fluids such as, but not limited to, whole blood, plasma/serum, dried blood spot, urine, sweat, saliva, sputum and cerebrospinal fluid. The sample is obtained from the subject in a manner well-established in the art.

In one embodiment, the subject is a human subject with no confounding disease, for example, the subject does not have diabetes and/or the subject is free of any condition which adversely affects kidney function, e.g. a condition which decreases kidney function by at least 10% or more, such as 25%, 50% or more.

Once a suitable biological sample is obtained, it is analyzed to determine the signal response, or the level or concentration of the selected biomarker(s) in the sample. Prior to analysis, the sample may be subject to processing such as extraction, filtration, centrifugation or other sample preparation techniques to provide a sample that is suitable for further analysis. For example, biological fluids may be filtered or centrifuged (e.g. ultracentrifugation) to remove solids and other components from the sample, e.g. proteins, to facilitate analysis. As one of skill in the art will appreciate, biomarker level may be determined using one of several techniques established in the art that would be suitable for detecting such biomarkers, e.g. polar metabolites, in a biological sample, including mass spectrometry, chromatographic techniques such as high performance liquid chromatography and gas chromatography, immunoassay or enzyme-based assays with colorimetric, fluorescence or radiometric detection. As one of skill in the art will appreciate, biomarkers may be analyzed directly or may be chemically altered or derivatized for analysis, e.g. fatty acids may be hydrolyzed. The sample may further be analyzed by comparison against stable-isotope internal standards.

In a preferred embodiment, biomarker detection using a mass spectrometry (MS)-based method is used. Suitable MS-based methods for use include direct infusion-mass spectrometry, electrospray ionization (ESI)-MS, direct infusion-tandem mass spectrometry (DI-MS), desorption electrospray ionization (DESI)-MS, direct analysis in real-time (DART)-MS, atmospheric pressure chemical ionization (APCI)-MS, electron impact (EI) or chemical ionization (CI), as well as MS-based methods coupled with a separation technique, such as liquid chromatography (LC-MS), gas chromatography (GC-MS), or capillary electrophoresis (CE-MS) or (NACE-MS) mass spectrometry, or coupled with enzyme assays, immunoassays and/or the use of related biosensors (antibodies, aptamers etc.) with optical or electrochemical detection.

The method includes the measurement of at least one metabolite as a specific biomarker for PAD in a biological sample. Preferably, the level of at least two or more biomarkers is determined to screen for or diagnose PAD, for example, the level of between two to fourteen biomarkers as part of a panel of top-ranked metabolites to enhance sensitivity and specificity, e.g. between two and ten biomarkers are determined in a sample, and more preferably, the level of two to five biomarkers are determined in a sample, for use to screen or diagnose PAD.

Once the level of the selected biomarker(s) is determined, the level is compared to a control level to determine the average fold-change (FC) difference and statistical significance (p-value) between the biomarker measured in patient relative to a non-PAD control level (e.g., the baseline level in healthy non-PAD individuals or populations). The level of some of the metabolite biomarkers is increased in PAD subjects in comparison to the non-PAD control level. Examples of biomarkers that exhibit increased levels in PAD subjects relative to non-PAD control subjects (i.e. fold-change>1) include creatinine, carnitine, propionylcarnitine, cystine, phenylacetylglutamine and trimethylamine-N-oxide. Other metabolite biomarkers exhibit a reduced level in PAD subjects in comparison to non-PAD control subjects (i.e., fold-change<1), such as histidine, creatine, lysine, tyrosine, monomethylarginine, oxo-proline, and the fatty acid biomarkers, e.g. stearic acid, linoleic acid, heptadecanoic acid, etc. The term “control level”, as it is used herein, is the level of a selected metabolite biomarker detected in a sample from a healthy non-PAD subject, preferably the control value is a mean control value obtained from a healthy population of matched subjects (e.g. age-, gender- and/or ethnically-matched to a population).

In one embodiment, at least one of the biomarkers detected in the method is selected from creatine, histidine, phenylacetylglutamine, tyrosine and oxoproline.

In another embodiment, at least two biomarkers are detected when one of the biomarkers is histidine, carnitine, TMAO, creatinine, tyrosine or the ratiometric biomarker, Phe/Tyr.

In addition to the quantitation of selected biomarkers, a ratiometric determination of two biomarkers may be calculated, i.e. the ratio of the levels of two biomarkers from a sample, for comparison against a control value, i.e. the ratio of the control levels of the two selected biomarkers. For example, the ratio of the level of the biomarker, phenylalanine, and the level of the biomarker, tyrosine, may be determined in a biological sample, and compared to a control ratio of the levels of these two biomarkers, to determine the difference between the ratio in the sample and the control ratio. Preferred ratiometric determinations for use in the present method are between a metabolite biomarker that exhibits an increased level in PAD, e.g. creatinine, carnitine, proprionylcarnitine, cystine or trimethylamine-N-oxide, and a metabolite biomarker that exhibits a reduced level in PAD, e.g. a fatty acid such as stearic acid, heptadecanoic acid, linoleic acid and arachidic acid. Such a ratio further amplifies the fold-change of the selected biomarkers and increases statistical significance (p value) for PAD screening or diagnosis, while also correcting for differences in sample volume in the biological sample analyzed.

In one embodiment, the ratiometric biomarker is selected from stearic acid:carnitine, and arginine:propionylcarnitine.

A human subject is determined to have PAD when the difference in the level of one or more biomarkers in a biological sample is statistically different from the control level of the corresponding biomarker, and/or when the difference in a ratiometric determination between two biomarkers is statistically different from the control ratio between these biomarkers above a minimum control threshold established for a population. The determination of statistical significance is well-established in the art. Statistical significance is attained when a p-value is less than the significance level. The p-value is the probability of observing an effect given that the null hypothesis is true whereas the significance or alpha (a) level is the probability of rejecting the null hypothesis given that it is true. Generally, a statistically significant difference, i.e. increase or decrease, in the level of a biomarker in accordance with the present method, is a difference in the level of the biomarker from the control level of at least about 5%, or greater, e.g. at least about 10%, 15%, 20%, 25% or more. When performing multivariate statistical analysis during biomarker discovery in metabolomics, corrected p-values are often used to correct for multiple hypothesis testing in order to reduce false discoveries, such as the use of a false discovery rate (q<0.05) or a more conservative Bonferroni correction.

In another aspect of the invention, a method of distinguishing chronic limb-threatening ischemia (CLTI) from intermittent claudication (IC) in a mammalian subject having PAD is provided. The method comprises: detecting in a biological sample from the subject the level of one or more metabolic biomarkers selected from the group consisting of: creatine, carnitine, propionylcarnitine, cystine, tyrosine, a fatty acid biomarker selected from the group consisting of stearic acid, linoleic acid, heptadecanoic acid, palmitic acid, oleic acid, heptadecenoic acid and pentadecanoic acid, and a ratiometric biomarker comprising at least one of the metabolic biomarkers; comparing the level of the one or more detected biomarkers to the corresponding level in an IC control; and determining that the subject has CLTI when the level of the one or more detected biomarkers is statistically different from the corresponding level in the IC control.

As used herein, the term “IC control” is the level of a given biomarker in a subject with IC, e.g. early-stage IC. Preferably, the IC control value is a mean control value obtained from an early-stage IC population of matched subjects (e.g. age-, gender- and/or ethnically-matched population). The term “early-stage PAD” or “IC” is defined clinically as intermittent leg pain, some impairment in walking, and mild to moderate blood flow blockage to lower limbs (ABI from 0.6 to 0.9). CLTI, also referred to as later stage of PAD progression or “late-stage PAD”, is defined clinically as continuous leg pain, severe impairment to walking and more severe blood flow blockage to lower limbs (ABI<0.5).

The level of some of the metabolite biomarkers is increased in CLTI subjects in comparison to the IC control level. Examples of biomarkers that exhibit increased levels in CLTI subjects relative the IC control level include creatinine, carnitine, propionylcarnitine and cystine. Other metabolite biomarkers exhibit a reduced level in CLTI subjects in comparison to IC control subjects such as the fatty acid biomarkers, e.g. stearic acid, linoleic acid, heptadecenoic acid, etc.

In one embodiment, at least one of the biomarkers detected in the method is selected from stearic acid, linoleic acid, heptanoic acid, creatinine, carnitine, propionylcarnitine and cystine.

In another embodiment, when two or more biomarkers is detected, only one of the biomarkers is a fatty acid biomarker.

In one embodiment, the biomarker is a ratiometric biomarker. Preferred ratiometric biomarkers are based on two biomarkers, one of which exhibits an increased level compared to the IC control level and the other of which exhibits a decreased level compared to the IC control level. In this regard, metabolite biomarkers that exhibit an increased level in CLTI include, for example, creatinine, carnitine, propionylcarnitine and cystine, and metabolite biomarkers that exhibit a reduced level in CLTI include fatty acid biomarkers such as stearic acid, heptadecanoic acid, linoleic acid and arachidic acid. In embodiments, the ratiometric biomarker may be selected from stearic acid:carnitine, and arginine:propionylcarnitine.

Following diagnosis of PAD, including CLTI or CI, the subject may be appropriately treated to manage symptoms, such as leg pain, and to prevent or reduce the progression of artherosclerosis to minimize the risk of a cardiovascular event. Thus, treatments may include one or more of: i) medication to treat leg pain by increasing blood flow to the limbs (e.g. by blood thinning and widening the blood vessels) such as cilostazol or pentoxifylline (Pentoxil); cholesterol-lowering medications to reduce heart attach risk such as a statin, e.g. pravastatin or lovastatin; high blood pressure medications, if applicable, such as an ACE inhibitor, e.g. lisinopril (Prinivil, Zestril), a calcium channel blocker, e.g. amlodipine besylate (Norvasc), an angiotension II receptor blocker such as Irbesartan (Avapro); medication to prevent blood clots such as daily aspirin therapy or another medication, such as clopidogrel (Plavix). If the PAD is determined to have progressed to IC or CLTI, then angioplasty, thrombolytic therapy or by-pass surgery may be appropriate. Lifestyle changes may also be prescribed, including increased exercise, particularly walking, cessation of smoking and an altered diet.

The present methods advantageously provide a means to screen for and diagnose PAD in asymptomatic patients in a cost-effective manner while providing the necessary sensitivity and improved specificity as compared to the conventional, potentially inconclusive ABI method, particularly in diabetic patients. The present metabolite-based biomarkers associated with PAD are readily measured by mass spectrometry as a multiplexed instrumental platform already available in most clinical laboratories. The present methods also enable differential diagnosis of PAD, and its progression to IC and CLTI.

In addition to use to diagnose PAD, the present methods may also be used to monitor disease progression and/or treatment response to therapy. In this regard, for a human subject determined to have PAD, the level of one or more of the metabolite biomarkers in a biological sample from the subject may be monitored throughout the course of treatment to determine the efficacy of the treatment and whether or not further intervention is required. The determination of the level of the metabolite biomarker is determined on at least two occasions. In this case, the difference in a first level of the biomarker from a control level (e.g. the level in a healthy non-PAD subject or population) is determined (a first difference) and compared to a subsequent difference (second difference) which is the difference between a subsequent determined biomarker level and the control level. If the difference in biomarker level increases over time (difference 1 is less than difference 2), this indicates that the disease is progressing (or treatment is not effective), while no change in the difference of biomarker levels over time indicates that the disease is not progressing (or treatment may be effective), and a decrease in the difference of biomarker levels over time indicates disease remission (or treatment is effective).

Embodiments of the invention are described in the following examples which are not to be construed as limiting.

### Definitions

Unless otherwise indicated, the definitions and embodiments described in this and other sections are intended to be applicable to all embodiments and aspects of the present application herein described for which they are suitable as would be understood by a person skilled in the art.

The term “peripheral artery disease” (PAD) refers to a form of atherosclerosis manifested in the lower extremities leading to a cascade of symptoms from insufficient blood flow, including impaired/painful walking, reduced functional capacity, ischemic myopathy, and recurrent skin lesions.

The term “intermittent claudication” (IC) refers to a progressed form of peripheral artery disease (PAD) which may be characterized with varying degrees of pain in leg muscles induced by walking.

The term “chronic limb-threatening ischemia” (CLTI) refers to late or end-stage peripheral artery disease (PAD) which may be characterized by rest pain, non-healing ischemic ulcers, and gangrene requiring limb amputation.

The term “subject” refers to a mammalian subject, including both human and non-human subjects, preferably a human subject.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

As used in this application, the singular forms “a”, “an” and “the” include plural references unless the content clearly dictates otherwise.

The term “and/or” as used herein means that the listed items are present, or used, individually or in combination. In effect, this term means that “at least one of” or “one or more” of the listed items is used or present.

### EXAMPLES

The following non-limiting examples are illustrative of the present application:

### Example 1

### Materials and Methods

Study cohort and design: This study was approved by the research ethics board at St. Michael's Hospital-University of Toronto (REB #16-375), and informed consent was obtained from all participants in accordance with the Declaration of Helsinki principles. PAD diagnosis and classification into IC and CLTI were made according to specialists' clinical examination and arterial ultrasound (US) [18]. Patients with CLTI “Rutherford stage>4” referred to vascular surgery ambulatory clinics or emergency department at St. Michael's Hospital (Toronto, ON) from June 2017 to August 2017, were requested to participate in this study. Exclusion criteria included all patients on anticoagulants, chemotherapy or biological anti-inflammatory agents. Patients diagnosed with sepsis, type 2 diabetes, systematic inflammatory disease or with active/history of any cancer or deep vein thrombosis were excluded as well. Moreover, patients with a 6-month history of acute coronary syndrome, heart failure, or uncontrolled arrhythmia as defined by American College of Cardiology, also failed to meet the inclusion criteria of this study [19]. A total of 128 consecutive ambulatory patients were initially recruited, however only 20 CLTI patients met the inclusion criteria and consented for this study. Upon acceptance to participate, CLTI patients were matched with non-diabetic IC cases or “Rutherford stage 1-3” and non-PAD controls in a ratio of 1:1:1 by age group and biological sex. To do so, during the months of January and February 2018, 20 IC patients and 20 non-PAD participants were recruited to match the CLTI cohort. PAD status was defined clinically as per the Rutherford classification, whereas non-PAD controls were defined as patients with cardiovascular risk factors alongside a normal arterial ultrasound of the lower limbs, and palpable distal pulses without a significant clinical history of claudication. However, after matching the cohorts, two CLTI patients later withdrew their consent and were not included in the final study.

Chemicals and Reagents: All chemicals were purchased from Sigma Aldrich (St. Louis, Mo., USA) unless otherwise stated. Stock solutions for internal standards and metabolite standards were prepared in deionized water from a Barnstead EASYpure® II LF system (Dubuque, Iowa, USA) for hydrophilic compounds and in methyl-tert-butyl ether (MTBE) for lipophilic fatty acids. Ultra-grade LC-MS solvents (acetonitrile, methanol, 2-isopropanol and water) purchased from Caledon Laboratories Ltd. (Georgetown, ON, Canada) were used to prepare sheath liquid solution for spray formation and aqueous or nonaqueous background electrolyte (BGE) for CE separations. All stock solutions for chemical standards were stored at 4° C. Nanosep® 3 k Omega™ ultrafiltration devices (Pall Life Sciences, Port Wash., N.Y., USA) were used for processing diluted serum samples with a 3 kDa molecular weight cut-off filter for protein removal.

Serum Creatinine Measurement by Jaffé Method: Measurement of serum creatinine was performed at St. Michael's Hospital using a modified Jaffe colorimetric assay on a Beckman Coulter AU system/analyzer (Beckman Coulter, Inc., CA, USA). Briefly, creatinine standard reagent (OSR6678) is added to an aliquot of serum, where serum creatinine reacts with picric acid under alkaline conditions to form a yellow-orange complex and the rate of change in absorbance at 520/800 nm is proportional to serum creatinine concentration to minimize other reacting/absorbing interferences [20]. Serum creatinine measurements were used for the assessment of glomerular filtration rate (GFR) and kidney function for all PAD patients. Serum creatinine measurements by the Jaffe colorimetric assay were compared with multisegment injection-capillary electrophoresis-mass spectrometry (MSI-CE-MS) using independent serum aliquots collected from the same participants.

Serum Sample Collection and Preparation: Fasting blood samples were collected and immediately centrifuged at 4° C. within 1 h after clotting at room temperature, where serum was separated, aliquoted and stored frozen at −80° C. Frozen serum was then thawed slowly on ice, vortexed for 30 s and aliquoted prior to ultrafiltration or liquid extraction. An aliquot of 50 μL of serum was diluted two-fold with ultra-grade LC-MS water containing 40 μM of two recovery standards, 3-chloro-L-tyrosine (Cl-Tyr) and 3-cyclohexylamino-1-propanesulfonic acid (CAPS), which were used for metabolomic analyses by MSI-CE-MS under positive and negative mode detection, respectively. The diluted serum was vortexed for 30 s, transferred to a pre-rinsed ultrafiltration device that was centrifuged at 14,000 g for 10 min to separate proteins from the serum filtrate used for analysis of polar/hydrophilic metabolites. Ultrafiltration devices were pre-rinsed with ultra-grade LC-MS water, centrifuged for 5 min at 14,000 g and air dried for about 20 min prior to first use to wash out background additives (e.g., lactic acid). Thereafter, serum filtrates (15 μL) were diluted one or two-fold with ultra-grade LC-MS containing three internal standards, 4-fluoro-L-phenylalanine (F-Phe), 2-napthalenesulfonic acid (NMS) and ¹³C₆-glucose. The final concentrations for all the internal and recovery standards were 10 μM with the exception of ¹³C₆-glucose (2 mM), and diluted serum filtrate samples were analyzed using MSI-CE-MS with two aqueous BGE systems optimal for separation of ionic/hydrophilic metabolites [21, 22].

A second aliquot of frozen serum was slowly thawed on ice and processed separately for lipid analysis following acid hydrolysis and liquid extraction as described previously [23, 24]. In this work, total (hydrolyzed) serum fatty acids were analyzed by multisegment injection-nonaqueous capillary electrophoresis-mass spectrometry (MSI-NACE-MS). Acid-catalyzed hydrolysis of esterified lipids was performed by addition of 25 μL of serum, 25 μL of 2.5 M sulfuric acid and 25 μL of 0.01% v/v butylated hydroxytouene (BHT) as an antioxidant additive in toluene followed by incubation at 80° C. for 1 h. Serum fatty acids were subsequently extracted using a slightly modified extraction protocol originally reported by Matyash et al. [25] using 500 μL of MTBE containing 50 μM of deuterated myristic acid (14:0-d27) as a recovery standard with 12.5 μL of 1 M HCl for better extraction efficiency. Following vigorous shaking for 30 min at room temperature, phase separation was then induced by addition of 100 μL of deionized water. Samples were then centrifuged at 3,000 g at 4° C. to sediment protein for 30 min resulting in phase separation into a water and ether (top) layer. A fixed volume (200 μL) was collected from the upper MTBE layer into a new vial then dried under a gentle stream of nitrogen gas at room temperature. Serum extracts were then stored dry at −80° C. and at time of analysis reconstituted in 25 μL of acetonitrile/isopropanol/water (70:20:10 v/v) with 10 mM ammonium acetate and 50 μM deuterated stearic acid (18:0-d35) as an internal standard. All hydrolysis reactions and extractions were carried out using glass GC vials that were pre-rinsed with dichloromethane and all pipette tips used during this procedure were pre-rinsed with methanol to minimize background palmitic acid (16:0) and stearic acid (18:0) contamination [23]. An internal quality control (QC) sample was prepared in-house by pooling together serum aliquots from all study participants. Separate QC aliquots were processed using the same sample protocols described above, stored at −80° C. and thawed once prior to analysis by MSI-NACE-MS using a nonaqueous BGE system optimal for separation of acidic lipids.

Hydrophilic Metabolome Profiling by MSI-CE-MS: MSI-CE-MS experiments were performed on an Agilent G7100A CE instrument (Agilent Technologies Inc., Mississauga, ON, Canada) equipped with a coaxial sheath liquid (Dual AJS ESI) Jetstream electrospray ionization source coupled to an Agilent 6550 quadrupole-time-of-flight (QTOF) system. An Agilent 1260 Infinity Isocratic pump equipped with a 100:1 splitter and a 1260 Infinity degasser were used to deliver the sheath liquid at a rate of 10 μL/min. Separations were performed using uncoated fused-silica capillaries (Polymicro Technologies, AZ, USA) of a total length of 130 cm and inner diameter of 50 μm. The BGE consisted of 1.0 M formic acid with 13% vol acetonitrile as organic modifier (pH 1.80) under positive ion mode, and 35 mM ammonium acetate (pH 9.50) under negative ion mode for comprehensive analysis of cationic and anionic serum metabolites, respectively [26-28]. Approximately 7 mm of the polyimide coating was removed from both the capillary inlet and the outlet using a capillary window maker (MicroSolv, Leland, N.C., United States) to reduce sample carry-over as well as polymer swelling or degradation when in contact with organic solvent or buffer solutions containing ammonia [29, 30]. Samples were injected hydrodynamically at 50 mbar (5 kPa) for 5 s for each sample interspaced with alternating BGE spacer plugs injected hydrodynamically for 40 s for positive mode and electrokinetically for 45 s at 30 kV for negative mode for a total of 7 discrete serum filtrate analyzed within a single run. Customized serial injection configurations using calibrant mixtures, filtrate blanks, and pooled QC samples were analyzed in MSI-CE-MS depending on study design requirements. Prior to first use, each bare fused-silica capillary was conditioned by flushing for 15 min at 950 mbar (95 kPa), sequentially, with methanol, 1.0 M sodium hydroxide, water, and BGE. An applied voltage of +30 kV at 25° C. was used for all CE separations under normal polarity, however a pressure gradient of 2 mbar/min was implemented for faster elution of slow migrating anions under negative ion mode conditions [29]. Between runs, the capillary was flushed with BGE for 15 min at 950 mbar (95 kPa). The sheath liquid was comprised of 60% vol MeOH with 0.1 vol % concentrated formic acid for positive ion mode, and 80% vol MeOH for negative ion mode detection. Reference ions purine and hexakis(2,2,3,3-tetrafluoropropoxy)-phosphazine (HP-921) were spiked into the sheath liquid at 0.02% v/v providing constant mass signals to enable real-time mass calibration and to allow for monitoring of potential ion suppression effects during separation. The instrument was operated in 2 GHz extended dynamic range. The Vcap and nozzle voltage were both set at 2000 V, while the fragmentor was 380 V, the skimmer was 65 V and the octopole RF was 750 V. The QTOF-MS system was operated with full-scan data acquisition over a mass range of m/z 50-1700 and an acquisition rate of 1 spectrum/s. At the beginning of each day, the QTOF-MS system was calibrated before analysis using an Agilent tune mixture to ensure residual mass ranges did not exceed 1 ppm. Additionally, daily cleaning of the CE electrode and ion source with 50% vol isopropanol was performed as preventative maintenance. A standard mixture run followed by a QC sample run with blank were analyzed at the start of each day to equilibrate the CE-MS system and assess system stability. Each serum filtrate sample from PAD and non-PAD participants in this study were analyzed in duplicate over two consecutive days by MSI-CE-MS under positive and negative ion modes for cationic/zwitter-ionic (e.g., amino acids) and anionic (e.g., organic acids) metabolites.

Lipophilic Metabolome Profiling by MSI-NACE-MS: Total serum (hydrolyzed) fatty acids were analyzed by MSI-NACE-MS under negative ion mode conditions as described elsewhere [23, 24]. An Agilent 6230 time-of-flight (TOF) mass spectrometer with a coaxial sheath liquid electrospray (ESI) ionization source equipped with an Agilent G7100A CE unit was used for all experiments (Agilent Technologies Inc., Mississauga, ON, Canada). An Agilent 1260 Infinity Isocratic pump and a 1260 Infinity degasser were applied to deliver an 80:20 vol methanol:water with 0.5% vol ammonium hydroxide at a flow rate of 10 μL/min using a coaxial sheath liquid interface kit. For real-time mass correction, reference ions purine and hexakis(2,2,3,3-tetrafluoropropoxy)phosphazine (HP-921) were spiked into the sheath liquid at 0.02% vol to provide constant mass signals at m/z 119.0363 and m/z 1033.9881, which were also used to monitor for potential ion suppression/enhancement effects during separation. The nebulizer spray was set off during serial sample injection but then subsequently turned on at a low pressure of 4 psi (27.6 kPa) following voltage application with the source temperature at 300° C. and drying gas delivered at 4 L/min. The instrument was operated in 2 GHz extended dynamic range with negative mode detection. Vcap was set at 3500 V while fragmentor was 120 V, the skimmer was 65 V and the Octopole RF was 750 V. Separations were performed on bare fused-silica capillaries with 50 μm internal diameter, 360 μm outer diameter and 95 cm total length (Polymicro Technologies Inc., AZ, USA), and a capillary window maker used to remove about 7 mm of the polyimide coating on both ends of the capillary. The applied voltage was set to 30 kV at 25° C. for CE separations together with an applied pressure of 20 mbar (2 kPa) used during the CE separation. The nonaqueous BGE was 35 mM ammonium acetate in 70% vol acetonitrile, 15% vol methanol, 10% deionized water, and 5% vol isopropanol with an apparent pH of 9.5 adjusted by addition of 12% vol of ammonium hydroxide. Serum extracts, pooled QC extracts, or fatty acid calibrants were injected hydrodynamically at 50 mbar (5 kPa) alternating between 5 s for each sample plug and 40 s for the BGE spacer plug for a total of seven discrete samples analyzed within one run. Prior to first use, capillaries were conditioned by flushing for 15 min at 950 mbar (95 kPa) sequentially with methanol, 0.1 M sodium hydroxide, deionized water, 1.0 M formic acid, deionized water then BGE for 30 min. Between runs, the capillary was flushed with BGE for 10 min at 950 mbar (95 kPa), and nonaqueous BGE and sheath liquid solutions were degassed before use.

Data Processing and Statistical Analysis: All MSI-CE-MS and MSI-NACE-MS data was analyzed using Agilent Mass Hunter Workstation Software (Qualitative Analysis, version B.06.00, Agilent Technologies, 2012). Molecular features were extracted in centroid and profile modes using a 10 ppm mass window for hydrophilic and lipophilic metabolites. Polar metabolites and hydrolyzed fatty acids were annotated based on their characteristic accurate mass (m/z) for their protonated, [M+H]+ or deprotonated, [M−H]− molecular ion together with their characteristic relative migration time (RMT), where apparent migration times were normalized to an internal standard migrating from the same sample position [26]. Extracted ion electropherograms (EIEs) were integrated after smoothing using a quadratic/cubic Savitzky-Golay function (15 points) and peak areas and migration times were transferred to Excel (Microsoft Office, Redmond, Wash., USA) for calculation of relative integrated peak area (RPA) and RMT. Data normalization to an internal standard (Cl-Tyr, CAPS, or 18:0-d35) improves precision in CE by correcting for differences in sample volumes introduced in-capillary, as well as migration time drift due to changes in electroosmotic flow between runs. Control charts for monitoring long-term method stability were derived from changes in measured responses (RPA) for recovery standards (F-Phe, NMS, or 14:0-d27) added to all serum filtrates or extracts, including QC samples. In most cases, serum metabolites and fatty acids were identified with high confidence (level 1) [31] using authentic standards with the exception for 8 metabolites (10% of total identified 85 metabolites; level 2 ID) where standards were unavailable. 7 unknown metabolites remain unidentified with only a putative molecular formula (level 4). Overall, 85 authentic metabolites were consistently detected (CV<30%) in the majority of serum samples (>75%) when applying stringent quality control (QC) measures on 800 initially extracted features to filter redundant and spurious signals arising from contaminants, artifacts, isotopes, in-source fragments, adducts or dimers.

Least-squares linear regression analysis for external calibration curves and control charts were performed using Excel (Microsoft Office, Redmond, Wash., USA). All multivariate data analysis, including principal component analysis (PCA), hierarchical cluster analysis (HCA), correlation matrix analysis (CMA), partial least-square discriminant analysis (PLS-DA), as well as receiver operating characteristic (ROC) curves were processed using MetaboAnalyst 4.0 (wwww.metaboanalyst.ca) [32], where data was normalized using a generalized log-transformation and autoscaling unless otherwise stated. PCA, HCA and CMA methods were used for data visualization (i.e., data trends, outlier detection), and comparing technical variance relative to overall biological variance, whereas PLS-DA was used for selecting significant serum metabolites associated with PAD progression. Additionally, ROC curves were performed only for the top-ranked ratiometric serum metabolites that discriminated between IC and CLTI based on the area under the curve (AUC) [32]. Baseline characteristics of PAD participants, including CON, IC, and CLTI sub-groups were compared using chi-square and Fisher exact tests for categorical variables and analysis of variance (ANOVA) for continuous variables. A one-way ANOVA was used to identify significant differences between CON, IC, and CLTI groups for all the metabolites with a polynomial contrasts analysis to identify linear trends associated with disease progression and Welch's F employed in case of inequality of variance tested by Levene's homogeneity test. This was followed by planned contrasts with contrast 1 comparing CON to all PAD cases [IC+CLTI], as well as contrast 2 comparing IC to CLTI (i.e., clinical PAD sub-groups), which was further confirmed by post-hoc analyses with Gabriel's and Games-Howell procedures. Also, a two-tailed student's t-test was employed on non-transformed and log-transformed data to compare CLTI to IC separately in the subgroup analysis while applying Benjamini Hochberg FDR correction (q<0.05) for multiple hypothesis testing. Pearson correlations were calculated to evaluate associations between the ABI and the metabolites on non-transformed data for the majority of variables and log-transformed data for non-normally distributed variables which was further confirmed with partial correlations adjusting for BMI and past smoking history. Normality tests Shapiro-Wilk test (p<0.05), Pearson and partial correlations, ANOVA, t-tests and nonparametric statistical analysis (Kruskal-Wallis and Mann-Whitney U test) were performed using the Statistical Package for the Social Science (IBM SPSS, version 18.0. NY, USA). MedCalc version 12.5.0 (MedCalc Software, Ostend, Belgium) was used for generation of boxplots as well as Bland-Altman % difference plot and Passing-Bablok regression used in the inter-laboratory method comparison of serum creatinine concentrations.

### Results and Discussion

Cohort Demographics and Clinical Characteristics: This study comprised a cohort of non-diabetic older persons (n=58) with an mean age of 63 years, including PAD patients at different stages of disease progression as clinically defined by the six-stage Rutherford classification (stages 1-3: IC, and stage≥4: CLTI), as well as non-PAD controls (CON). A summary of the study demographics and other clinical characteristics for participants is summarized in Table 1. There were no significant differences in age, sex, body mass index (BMI), glycated haemoglobin, white blood cell, and platelet counts between the three patient sub-groups. The IC and CLTI patients presented with higher incidence of dyslipidemia, coronary artery disease, and statin/antiplatelet medication use than controls. However, all patients with a 6-month history of acute coronary syndrome, heart failure, or uncontrolled arrhythmia were excluded in this study eliminating active coronary symptoms at the time of sampling. Importantly, when analyzing differences between the two PAD subgroups, IC and CLTI, patients were closely matched with no statistical differences (p>0.05) in anthropometric properties, comorbidities, or medication use with the exception for ABI (p=3.06×10⁻³³), which was lower in CLTI (mean ABI=0.38) as compared to IC (mean ABI=0.57) and non-PAD controls (mean ABI=1.08).

TABLE 1
Baseline patient demographics/clinical characteristics for PAD
sub-groups (IC, CLTI) and non-PAD controls (CON)

| | CON | IC | CLTI | |
|---|---|---|---|---|
| Parameter | (n =20) | (n = 20) | (n = 18) | p-value |
| Rutherford | — | 1-3 | ≥4 | 3.06 × 10⁻³³; |
| stage | | [2.75 ± 0.4] | [4.11 ± 0.3] | 2.36 × 10⁻⁹ |
| Walking | >1000 | 530 | <160 | 0.151; 0.055 |
| distance (m) | | | | |
| ABI | 1.08 ± 0.09 | 0.57 ± 0.08 | 0.38 ± 0.07 | 0.061; 0.631 |
| Age (years) | 62.6 ± 6.6 | 61.0 ± 7.4 | 65.2 ± 5.6 | 0.217; 0.124 |
| BMI (kg/m2) | 26.6 ± 2.5 | 24.3 ± 3.0 | 24.9 ± 3.6 | 0.156; 0.534 |
| HbA1c (%) | 5.75 ± 0.51 | 5.98 ± 0.50 | 5.58 ± 0.99 | 0.152; 0.118 |
| WBC | 6.6 ± 2.2 | 7.8 ± 2.5 | 8.4 ± 3.4 | 0.401; 0.224 |
| Platelets | 251 ± 076 | 244 ± 64 | 209 ± 65 | 0.002; 0.730 |
| Males (%) | 50 (10/20) | 55 (11/20) | 72 (13/18) | — |
| Smoking (%) | 55 | 95 | 94 | 0.099; 0.450 |
| Diabetes | 0 | 0 | 0 | 0.001; 0.616 |
| mellitus (%) | | | | |
| Hypertension | 40 (8/20) | 65 (13/20) | 72 (13/18) | 0.76; 0.730 |
| (%) | | | | |
| Hyper- | 39 (7/20) | 85 (17/20) | 83 (15/18) | 0.001; 0.165 |
| lipidaemia | | | | |
| (%) | | | | |
| Renal | 0 (0/20) | 5 (1/20) | 6 (1/18) | <0.001; 0.066 |
| insufficiency | | | | |
| (%) | | | | |
| Coronary | 0 (0/20) | 40 (8/20) | 61 (11/18) | <0.001; — |
| artery | | | | |
| disease (%) | | | | |
| Statin use (%) | 30 (6/20) | 80 (16/20) | 100 (18/18) | 3.06 × 10⁻³³; |
| | | | | 2.36 × 10⁻⁹ |
| Antiplatelet | 50 (10/20) | 100 (20/20) | 100 (18/18) | 0.151; 0.055 |
| use (%) | | | | |

Data shown as mean ± standard deviation for continuous variables and % (number of cases/total) for categorical variables.
p-value represents overall difference between the three groups where significant differences are observed for (p < 0.05) calculated Fisher exact tests for categorical variables and ANOVA for continuous variables; followed by p-value for PAD subgroup comparison between CLTI and IC calculated using independent samples t-test or non-parametric Mann-Whitney U test (only for BMI). Smoking reflects numbers of past/current smokers.

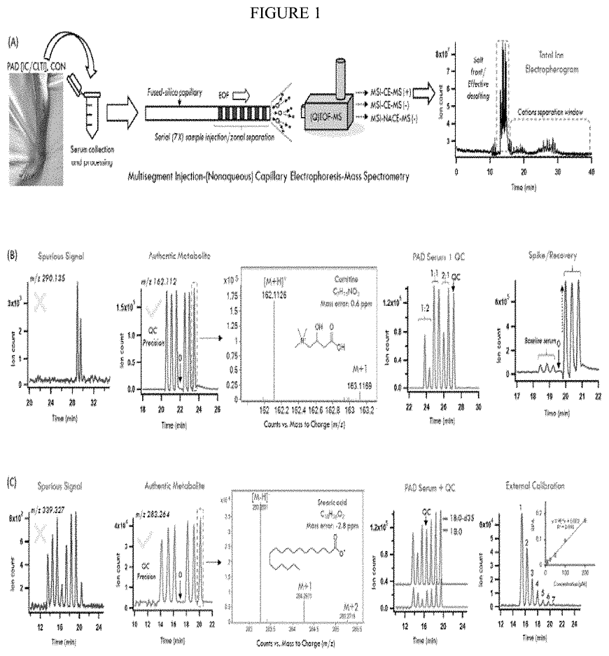

The Serum Metabolome of PAD Patients: Nontargeted characterization of the serum metabolome was performed by MSI-CE-MS and MSI-NACE-MS for polar/hydrophilic and non-polar/lipophilic metabolites, respectively. Serum samples from CON, IC and CLTI patient sub-groups were randomly analyzed in duplicate (except for fatty acids that used a single analysis) together with a pooled serum sample as a QC to monitor for long-term signal drift, as well as authenticate metabolites based on their characteristic accurate mass and relative migration time under positive or negative ion mode (m/z:RMT:p/n). For instance, serum metabolites were readily identified in a single run when using multiplexed separations with temporal signal pattern recognition with full data acquisition provided that their ion responses were measured with adequate precision (CV<30%, n=6) with no signal detected in blank filtrate/extract as depicted in FIG. 1. This process was used to reject spurious signals, background artifacts, as well as redundant ions derived from same metabolite (e.g., in-source fragments, isotopic signals, and adducts) that can constitute a majority of signals (>90%) generated in ESI-MS. Also, the identity of most serum metabolites was confirmed using authentic standards (level 1) based on their co-migration (RMT<1%) with low mass error (<5 ppm), which were also used for their quantification. Otherwise, 7 serum metabolites with unknown chemical structures were annotated based on their most likely molecular formula (level 4). Serum metabolites were reported in this work only if they satisfied two additional inclusion criteria to reduce false discoveries win metabolomics, namely they were measured with adequate precision throughout the study (CV<30% from QC runs) and detected with high frequency (>75% samples). This iterative process of data filtering and selection culminated in a final data matrix of 85 serum metabolites reliably measured in the majority of samples in this cohort, including 42 hydrophilic cations, 18 hydrophilic anions and 25 lipophilic anions. Overall, the serum metabolome coverage included a diverse range of circulating metabolites ranging from amino acids, amines, organic acids, long-chain fatty acids, ketone bodies, hexoses, and osmolytes/uremic toxins.

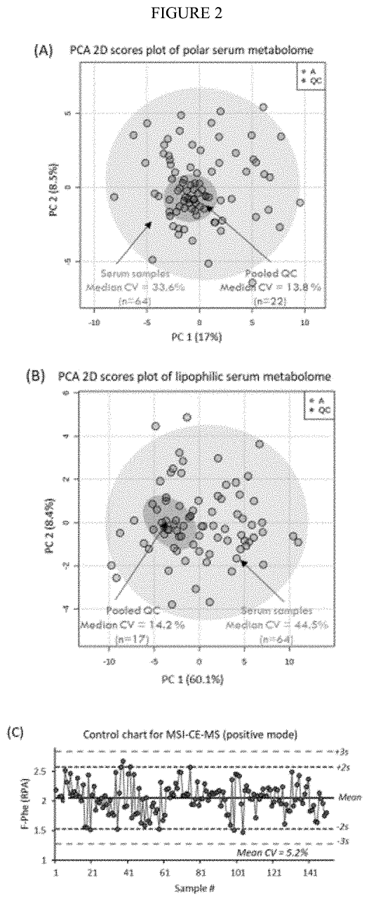

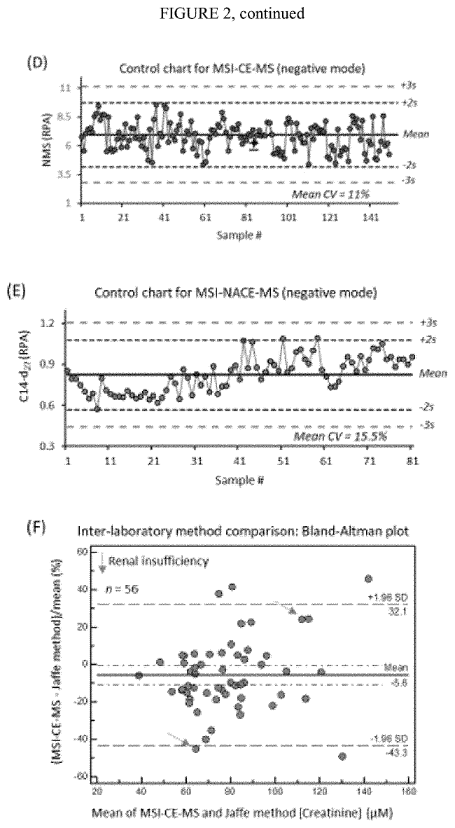

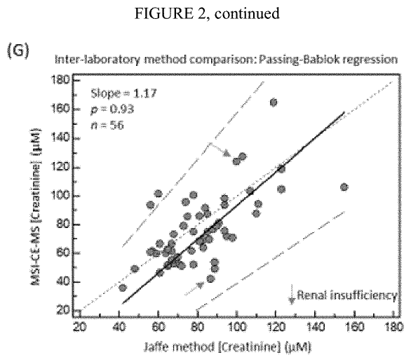

As expected, the overall biological variance of the serum metabolome (median CV=39%, n=58) was considerably larger than the technical precision of the method based on repeated analysis of a QC sample in each run (median CV=14%, n=39) as shown in the 2D scores plots from PCA in FIGS. 2A and B. Also, control charts for the three recovery standards used in MSI-CE-MS (10 μM F-Phe and NMS) and MSI-NACE-MS (50 μM 14:0-d27) added to all serum samples prior to ultrafiltration or MTBE extraction further demonstrate acceptable intermediate precision (mean CV<15%) with few outliers (<2%, n=57 total runs) exceeding warning limits (±2 s) (FIGS. 2C, D, E). An inter-laboratory method comparison of serum creatinine concentrations measured independently from 56 participants was also performed by MSI-CE-MS relative to the Jaffe colorimetric method, which was used for estimation of GFR for patients as an indicator of kidney function [20]. In this case, Bland-Altman % difference plot confirms good mutual agreement for serum creatinine determination by both methods with a mean bias of −5.6% (FIG. 2F). Also, there was a random distribution in the data with few outliers (4 of 56) exceeding agreement limits (±2 s). Similarly, a Passing-Bablok regression analysis (FIG. 2G) reveals no statistically significant difference from the line of equality (p=0.93) with a modest positive slope of 1.17. Only 2 PAD patients (1 IC and 1 CLTI) out of the 58 study participants overall were diagnosed with renal insufficiency as indicated by arrows.

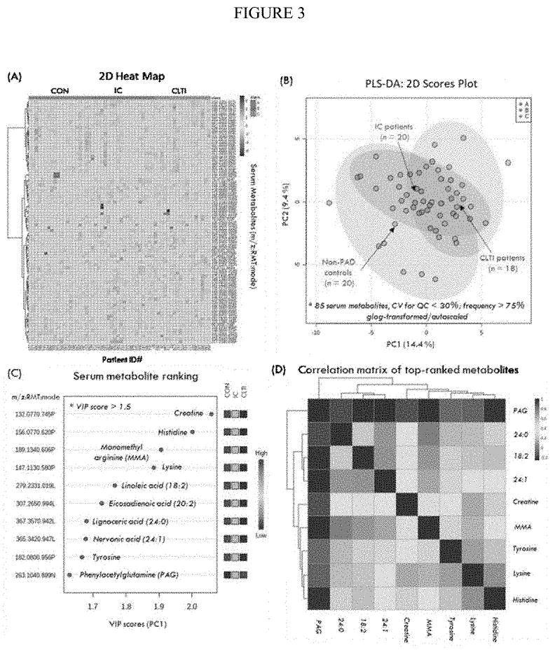

Differentiating Serum Metabolites in PAD Patients: FIG. 3A depicts a 2D heat map with HCA that summarizes the overall data structure involving 85 serum metabolites consistently measured in all 58 study participants (with only 0.7% missing values), including CON, IC, and CLTI sub-groups. FIG. 3B depicts a 2D scores plot from PLS-DA for differentiation of the metabolic phenotype in CLTI patients (n=18) from matched IC patients (n=20), as well as CON (n=20) based on glog-transformed and autoscaled data. FIG. 3C summarizes ten top-ranked serum metabolites largely responsible for group separation along the first principal component (variable importance in projection, VIP>1.5), including creatine (Crt), lysine (Lys), histidine (His), monomethylarginine (MMA), tyrosine (Tyr), phenylacetylglutamine (PAG), and several long-chain fatty acids (18:2, 20:2, 23:0, 24;1). FIG. 3D depicts a correlation matrix for the top-ranked serum metabolites associated with PAD progression, which highlights two major clusters of strongly co-linear serum metabolites not correlated to PAG, namely amino acids (r>0.60 with Lys), and fatty acids (r>0.70 with linoleic acid, 18:2). Univariate statistical analysis was also performed to confirm the significance of serum metabolites associated with PAD progression when using a one-way ANOVA with planned contrasts as summarized in Table 2. Overall, 14 serum metabolites were determined as significant (p<0.05) when using a linear contrast analysis model across all three categories (i.e., CON-IC-CLTI), including 10 metabolites identified by the PLS-DA model, as well as 4 additional metabolites, including oxo-proline (oxo-Pro), behenic acid (23:0), creatinine, and cystine. Also, phenylalanine:tyrosine ratio (Phe/Tyr) was higher in CLTI as compared to IC as it was reported an indicator of inflammation in PAD [16]. Importantly, most serum metabolites exhibited a linear change in their concentrations (or RPAs) as a function of PAD status (p<0.05). Overall, discrimination between the three PAD sub-groups follows a linear trend where IC clusters in the middle between CON and more severe CLTI cases similar to the 2D scores plot in PLS-DA.

TABLE 2
Top-ranked serum metabolites which reflect disease progression

| | | | | | | p- | | p- | | | |
|---|---|---|---|---|---|---|---|---|---|---|---|
| | | | p- | | | value | | value | | r | |
| | | | value | p- | | Cont1 | FC | Cont. 2 | FC | correlat | p- |
| | | F- | over- | value | Effect | PAD: | PAD: | CLTI: | CLTI: | to | value |
| Metabolite ID | m/z:RMT:mode | value | all | linear^{a} | size^{b} | CON^{c} | CON^{c} | IC^{d} | IC^{d} | ABI^{c} | for r |
| Creatine | 132.077:0.745:p | 6.02* | 0.006 | 0.002 | 0.422 | 0.008 | 0.65 | 0.097 | 0.75 | 0.44 | 0.001 |
| HMDB000064 | | | | | | | | | | | |
| Histidine | 156.077:0.620:p | 5.54 | 0.006 | 0.003 | 0.410 | 0.002 | 0.85 | 0.435 | 0.95 | 0.38 | 0.004 |
| HMDB000117 | | | | | | | | | | | |
| Phenylacetyl- | 263.104:0.899:n | 5.43* | 0.009 | 0.017 | 0.319 | 0.030 | 1.89 | 0.880 | 0.94 | −0.30 | 0.020f |
| glutamine | | | | | | | | | | | |
| HMDB0006344 | | | | | | | | | | | |
| Lysine | 147.113:0.580:p | 4.23 | 0.020 | 0.005 | 0.365 | 0.014 | 0.85 | 0.137 | 0.88 | 0.35 | 0.007f |
| HMDB0000182 | | | | | | | | | | | |
| Tyrosine | 182.080:0.956:p | 3.53 | 0.036 | 0.014 | 0.338 | 0.012 | 0.81 | 0.520 | 0.94 | 0.34 | 0.008f |
| HMDB0000158 | | | | | | | | | | | |
| Monomethyl-arginine | 189.134:0.606:p | 3.19 | 0.049 | 0.022 | 0.332 | 0.005 | 0.74 | 0.378 | 0.93 | 0.32 | 0.014f |
| HMDB0029416 | | | | | | | | | | | |
| Oxo-proline | 128.035:1.137:n | 2.89 | 0.054 | 0.028 | 0.316 | 0.021 | 0.69 | 0.638 | 0.86 | 0.34 | 0.013 |
| HMDB0000267 | | | | | | | | | | | |
| Creatinine | Jaffé method | 6.57 | 0.003 | 0.002 | 0.446 | 0.055 | 1.27 | 0.003 | 1.25 | −0.31 | 0.020 |
| Creatinine | 114.066:0.614:p | 6.14 | 0.004 | 0.011 | 0.428 | 0.271 | 1.07 | 0.001 | 1.30 | −0.30 | 0.035g |
| HMDB0000562 | | | | | | | | | | | |
| Linoleic acid (18:2n-6) | 279.233:1.019:1 | 4.96 | 0.010 | 0.007 | 0.390 | 0.101 | 0.78 | 0.009 | 0.95 | 0.24 | 0.066 |
| HMDB0000673 | | | | | | | | | | | |
| Eicosadienoic acid | 307.265:0.994:1 | 4.30 | 0.018 | 0.010 | 0.368 | 0.089 | 0.79 | 0.019 | 1 | 0.25 | 0.061 |
| (20:2) HMDB0005060 | | | | | | | | | | | |
| Nervonic acid (24:1) | 365.342:0.947:1 | 3.96 | 0.025 | 0.012 | 0.356 | 0.095 | 0.86 | 0.026 | 0.75 | 0.23 | 0.085 |
| HMDB0002368 | | | | | | | | | | | |
| Phe/Tyr | — | 3.67 | 0.032 | 0.026 | 0.343 | 0.22 | 1.10 | 0.018 | 1.15 | −0.25 | 0.055 |
| Behenic acid (22:0) | 339.327:0.969:1 | 3.49 | 0.038 | 0.026 | 0.336 | 0.187 | 0.91 | 0.024 | 0.75 | 0.22 | 0.105 |
| HMDB0000944 | | | | | | | | | | | |
| Lignoceric acid (23:0) | 367.358:0.942:1 | 3.45 | 0.037 | 0.015 | 0.334 | 0.088 | 0.84 | 0.045 | 0.75 | 0.26 | 0.050 |
| HMDB0002003 | | | | | | | | | | | |
| Cystine | 241.030:0.933:p | 3.15* | 0.050 | 0.028 | 0.377 | 0.305 | 1.07 | 0.019 | 1.29 | −0.24 | 0.065 |
| HMDB0000574 | | | | | | | | | | | |

Abbreviations:
p: positive aqueous mode,
n: negative aqueous mode,
l: negative non-aqueous mode,
CON: non-PAD controls,
PAD: peripheral artery disease (IC + CLTI),
CLTI: chronic limb-threatening ischemia,
IC: matched intermittent claudication,
FC: fold-change,
ABI: ankle-brachial index;
*Welch's F employed in case of inequality of variance tested by Levene's homogeneity test;
^{a}p-value for a linear trend when applying polynomial contrasts analysis;
^{b}Effect size calculated based on eta-squared;
^{c}p-value and mean FC for planned contrast 1 comparing PAD to CON;
^{d}p-value and mean FC for planned contrast 2 comparing CLTI to IC;
^{e}Pearson correlation on normally-distributed non-transformed or log-transformed serum metabolites to ABI after adjusting for Bill and smoking.
^{f}not significant after adjusting for Bill and smoking.

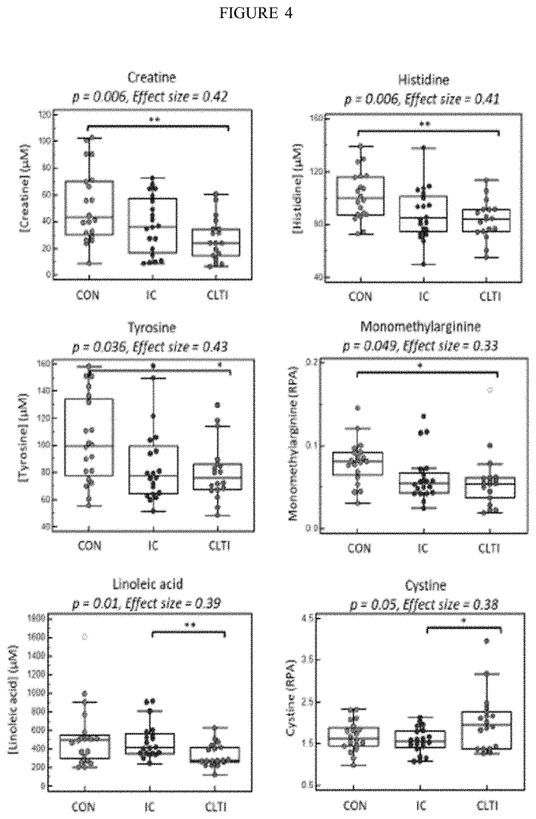

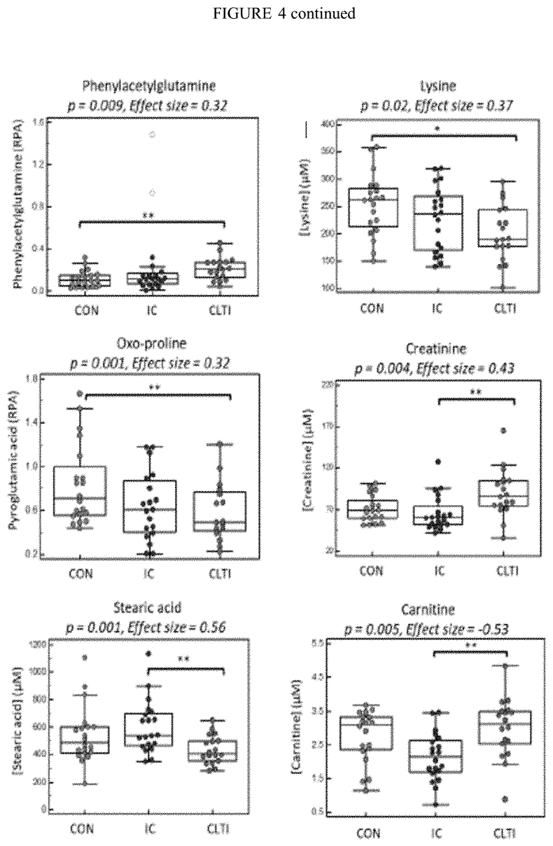

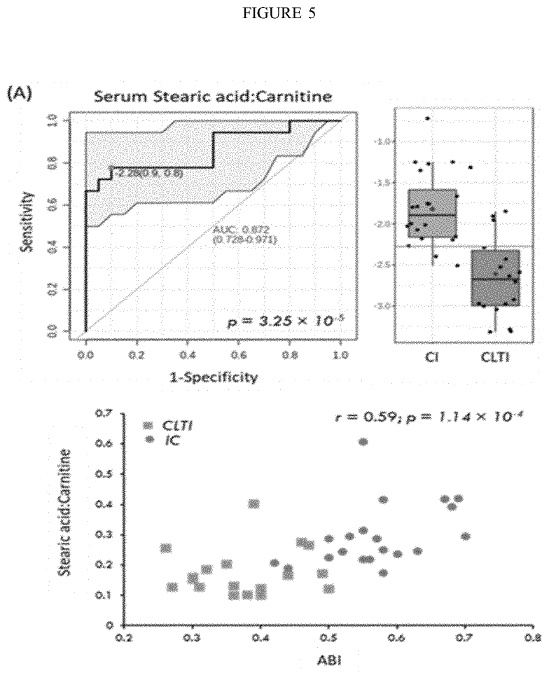

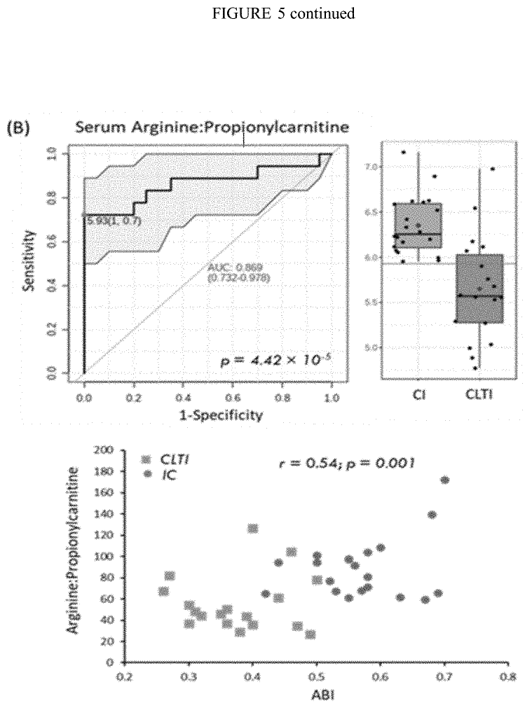

Further analysis was next performed to identify specific between-group differences without inflating type I error by using an ANOVA with two discrete planned contrasts, namely CON-PAD (contrast 1) and CLTI-IC (contrast 2). The box-whisker plots in FIG. 4 show that circulating concentrations of Crt, His, oxo-Pro, Lys, Tyr and MMA were higher in non-PAD controls as compared to PAD patients [IC+CLTI] unlike PAG (contrast 1, p<0.05). Furthermore, serum creatinine, cystine, and Phe/Tyr were elevated in CLTI patients as compared to IC cases, whereas a series of circulating fatty acids (18:2, 22:0, 20:2, 24:0, 24:1) display the opposite trend. As expected, a similar outcome was found for serum creatinine measured by the Jaffe method confirming good mutual agreement with MSI-CE-MS results. An additional subgroup analysis using a two-tailed student's t-test was deemed appropriate to better evaluate PAD progression since IC and CLTI patients were closely matched in terms of age, sex, BMI, current smoking, co-morbidities and medication use as shown in Table 1. In this case, Table 3 summarizes that 16 serum metabolites were differentially expressed (p<0.05) in the two PAD sub-groups, including 11 metabolites after FDR adjustment (q<0.05). In this case, serum creatinine, carnitine (CO), propionylcarnitine (C3), cystine, Phe/Tyr, and trimethylamine-N-oxide (TMAO) were elevated in CLTI as compared to IC cases in contrast to several different fatty acids, including saturated/odd-chain fatty acids (15:0, 16:0, 17:0, 18:0). Interestingly, all putative serum biomarkers associated with PAD progression were also correlated with ABI that is used for risk stratification of symptomatic PAD patients, notably stearic acid (18:0, r=0.51, p=0.001), as well as CO and cystine (r=−0.48, p=0.002). Lastly, ROC curve analysis was also performed on all serum metabolites and their ratios to demonstrate reliable discrimination of high risk CLTI from lower risk IC patient sub-groups. FIG. 5 shows two top-ranked ratiometric biomarkers in serum with an AUC˜0.87 along with their 95% confidence intervals (0.73-0.98), namely 18:0/CO and arginine (Arg)/C3. These two ratiometric biomarkers also exhibit strong linear correlation with ABI from PAD patients (r=0.54 to 0.59, p<0.001, n=38). This is relevant for biomarker discovery in pilot studies in order to anchor aberrant metabolism to a validated physiological measure of clinical significance to PAD.

TABLE 3
Top-ranked serum metabolites comparing IC (n = 20) to CLTI (n = 18)
using student's t-test and their correlation to ABI

| | | | FDR | FC _{b} | r correlation | p-value |
|---|---|---|---|---|---|---|
| Metabolite ID | m/z:RMT:mode | p-value* | q-valueₐ | (CLTI/IC) | to ABI _{c} | for r |
| Stearic acid; | 283.264:1.005:1 | 0.001 | 0.014 | 0.72 | 0.51 | 0.001 |
| 18:0 | | | | | | |
| HMDB0000827 | | | | | | |
| Linoleic acid; | 279.233:1.019:1 | 0.003 | 0.028 | 0.68 | 0.39 | 0.016 |
| 18:2n-6 | | | | | | |
| HMDB0000673 | | | | | | |
| Heptadecanoic | 269.249:1.030:1 | 0.003 | 0.029 | 0.72 | 0.43 | 0.007 |
| acid; 17:0 | | | | | | |
| HMDB0002259 | | | | | | |
| Palmitic acid; | 255.233:1.030:1 | 0.004 | 0.030 | 0.73 | 0.37 | 0.024 |
| 16:0 | | | | | | |
| HMDB0000220 | | | | | | |
| Creatinine | 114.066:0.614:p | 0.004 | 0.031 | 1.30 | −0.45 | 0.004 |
| HMDB0000562 | | | | | | |
| Carnitine | 162.112:0.719:p | 0.005 | 0.031 | 1.28 | −0.48 | 0.002 |
| HMDB0000062 | | | | | | |
| Oleic acid; | 281.249:1.013:1 | 0.005 | 0.031 | 0.71 | −0.04 | 0.756 |
| 18:1n-9 | | | | | | |
| HMDB0000207 | | | | | | |
| Heptadecenoic | 267.233:1.026:1 | 0.008 | 0.043 | 0.73 | -0.01 | 0.961 |
| acid; 17:1n-9 | | | | | | |
| HMDB0062437 | | | | | | |
| Propionyl- | 218.138:0.784:p | 0.008 | 0.043 | 1.37 | 0.09 | 0.507 |
| carnitine | | | | | | |
| HMDB0000824 | | | | | | |
| Eicosadienoic | 307.265:0.994:1 | 0.009 | 0.047 | 0.72 | 0.37 | 0.023 |
| acid; 20:2n-6 | | | | | | |
| HMDB0005060 | | | | | | |
| Pentadecanoic | 241.217:1.042:1 | 0.010 | 0.047 | 0.66 | 0.33 | 0.044 |
| acid; 15:0 | | | | | | |
| HMDB0000826 | | | | | | |
| Cystine | 241.0299:0.933:p | 0.014 | 0.061 | 1.29 | −0.48 | 0.002 |
| HMDB0000574 | | | | | | |
| Arachidic acid; | 311.296:0.981:1 | 0.015 | 0.061 | 0.68 | 0.39 | 0.015 |
| 20:0n-3 | | | | | | |
| HMDB0002212 | | | | | | |
| Trimethylamine- | 76.077:0.544:p | 0.019 | 0.080 | 1.60 | −0.44 | 0.005 |
| N-oxide | | | | | | |
| HMDB0000925 | | | | | | |
| Nervonic acid; | 365.342:0.947:1 | 0.024 | 0.091 | 0.75 | 0.29 | 0.083 |
| 24:1 | | | | | | |
| HMDB0002368 | | | | | | |
| Phe/Tyr ratio | — | 0.022 | 0.103 | 1.19 | −0.33 | 0.041 |

Two-tailed exact p-value on log-transformed serum metabolome data.
ₐFDR correction for multiple hypothesis testing;
_{b} mean fold-change ratio when comparing relative ion response ratio for each metabolite as a ratio of CLTI/IC;
_{c} Pearson correlation on normally-distributed non-transformed or log-transformed data.

The lack of PAD awareness among physicians continues to pose a major diagnostic challenge due to its variable clinical manifestations and unpredictable aggressive progression. An improved screening strategy for early detection of PAD in asymptomatic patients is required given the poor sensitivity of specialized Doppler methods for ABI assessment at early stages of atherosclerosis in peripheral tissue [35]. In this case, prognostic biomarkers will augment well-established traditional risk factors (e.g., smoking, diabetes, age, hyperlipidemia, renal dysfunction) while allowing for reliable diagnosis of PAD especially in high-risk patients with calcified vascular tissue not suitable for ABI. Regrettably, blood-based protein biomarkers (e.g., inflammatory cytokines, C-reactive protein) have yet to be clinically validated for routine screening of PAD, predict disease progression, and/or monitor treatment responses of patients [6]. In this study, a panel of serum metabolites associated with PAD progression was identified, which is important given the poor survivorship of patients with CLTI following invasive surgical interventions, including revascularization procedures and limb loss from amputation.

Untargeted metabolite profiling was performed on fasting serum samples collected from PAD patients, including well-matched CLTI and IC cases, and non-PAD controls. Overall, 85 serum metabolites were consistently detected in the majority of serum samples with good technical precision when using three different configurations in MSI-(NA)CE-MS. This multiplexed separation method takes advantage of unique data workflows and iterative data filtering processes to authenticate metabolites from spurious, background and redundant signals in conjunction with stringent QC to reduce false discoveries. A panel of serum metabolites that differentiated CLTI from IC patients was identified, as well as PAD from non-PAD controls. Serum Crt was found to be one of the most significant metabolites lower in PAD as compared to CON (F-value=6.0, p=0.006, effect size=0.42), which also exhibited a linear change in concentration (p=0.002) across the three study sub-groups.

Additionally, lower antioxidant capacity within ischemic muscle tissue likely plays a role in the 0.83-fold reduction of serum His concentrations in PAD as compared to CON (F-value=5.5, p=0.006, effect size=0.41). Similar to trends identified in metabolite trajectories for His and Crt, PAD patients exhibit a decrease in serum Lys, MMA and oxo-Pro concentrations relative to non-PAD controls (Table 2, FIGS. 3 and 4). The 0.63-fold decrease of serum oxo-Pro levels in PAD cases as compared to non-PAD controls may indicate glutathione depletion and activation of the glutathione salvage pathway that is a hallmark of deleterious oxidative stress. This is consistent with elevated serum cystine in CTLI as compared to IC (p=0.014), which is a biomarker of systemic oxidative stress prevalent in PAD that is associated with adverse clinical outcomes independent yet synergistic to inflammation. An opposing trend was found for serum PAG that exhibited a 1.9-fold higher concentration in PAD as compared to CON (F-value=5.4, p=0.009, effect size=0.32). This circulatory uremic toxin is a strong and independent risk factor for cardiovascular disease, where elevated serum PAG may serve as predictor of overall mortality in high-risk patients with chronic kidney disease [43], but it has not been reported in PAD patients without renal dysfunction.

These results also indicate aberrant circulating lipid metabolism when comparing IC to CLTI patients in terms of several serum fatty acids (total hydrolyzed) that were highly co-linear (FIG. 3D) and consistently reduced in PAD as compared to CON (Table 2), notably when comparing CLTI to IC (Table 3). Overall, serum 18:0 was most significantly depleted in CLTI as compared to IC patients even after FDR adjustment (q<0.05) that was also strongly correlated with ABI (r=0.51, p=0.01). Mounting evidence supports that increasing circulating 18:0 lipids are associated with reduced blood pressure and improved heart function, which also promotes mitochondrial fusion with greater beta-oxidation activity [49] likely impaired among late-stage PAD patients with severe muscle ischemia.

Due to potential confounding factors when comparing PAD patients to non-PAD controls, several serum metabolites were significantly differentiated (most satisfying a FDR adjustment, q<0.05) despite comparing well-matched IC from CLTI patients (Table 3). A mean 1.3-fold increase in serum carnitine (p=0.005) likely reflects severe myofiber degeneration occurring in CLTI as compared to less acute muscle atrophy in IC. A higher degree of atherosclerosis associated with CLTI as compared to IC is consistent with elevated serum TMAO concentrations (p=0.019) in the PAD sub-group analysis. Crt deficiency in the circulation indicates a state of diminished energy supply in CLTI possibly contributing to disease progression. Additionally, Crt undergoes spontaneous irreversible non-enzymatic degradation within skeletal muscle tissue into creatinine, which diffuses out of the muscle tissue into the circulation to be excreted by glomerular filtration through the kidneys. In this work, a mean 1.3-fold increase in serum creatinine was measured in CLTI as compared to IC (p=0.004), which was replicated independently on two different instrumental platforms/laboratories when using a Jaffe colorimetric assay and MSI-CE-MS (FIG. 1G; Table 2). This finding highlights the consequence of ischemic muscle with advanced CLTI resulting in elevated serum creatinine, which was reported to predict mortality in PAD patients with renal failure independent of hypertension and diabetes [53]. Lastly we observed a higher serum Phe/Tyr in CLTI relative to IC. Given the need for improved risk stratification of PAD, other ratiometric biomarkers were found to be superior to Phe/Tyr as a diagnostic biomarker of CLTI (FIG. 5). In this case, both serum 18:0/CO and Arg/C3 display good accuracy in differentiating CLTI from IC patients with an AUC=0.870 (p=4.0×10-5) from ROC curves. As expected, these ratiometric serum biomarkers were also strongly correlated (r=0.54 to 0.59, p<0.001) with ABI and thus may prove useful for monitoring of PAD progression and/or enable screening of asymptomatic patients for early detection of PAD. This panel of serum metabolites offer a convenient approach for diagnostic testing as compared to ABI that may be used to predict CLTI risk while guiding optimal dietary, pharmacological and/or surgical interventions to mitigate PAD progression and improve long-term patient survivorship and quality of life.

In summary, this work is the first to elucidate robust metabolic signatures associated with PAD in age/sex-matched nondiabetic patients largely without renal failure or differences in prescribed medication use (e.g., statins, antiplatelets). A high throughput metabolomics platform was applied for nontargeted analysis of circulating metabolites and lipids from fasting serum samples while applying a rigorous approach to data filtering to reduce false discoveries. Importantly, it was revealed for the first time that lower serum creatine, monomethylarginine, oxo-Pro, and several fatty acids are indicative of CLTI as compared to IC and/or non-PAD controls with opposing trends for the uremic toxin, PAG. In addition, serum 18:0/CO and Arg:C3 were determined to be strong biomarkers of PAD that accurately differentiate CLTI from IC while also displaying strong correlations to ABI.

Relevant portions of publications referred to herein are incorporated by reference in their entirety to the same extent as if the relevant portion was specifically and individually indicated to be incorporated by reference in its entirety. Where a term in the present application is found to be defined differently in a document incorporated herein by reference, the definition provided herein is to serve as the definition for the term.

### FULL CITATIONS FOR DOCUMENTS REFERRED TO IN THE APPLICATION

1. Hirsch, A. T.; Criqui, M. H.; Treat-Jacobson, D.; Regensteiner, J. G.; Creager, M. A.; Olin, J. W.; Krook, S. H.; Hunninghake, D. B.; Comerota, A. J.; Walsh, M. E.; et al. Peripheral arterial disease detection, awareness, and treatment in primary care. JAMA 2001, 286, 1317-1324.
2. Conte M. S.; Bradbury A. W.; Kolh P.; White J. V.; Dick F.; Fitridge R.; Mills J. L.; Ricco J. B.; Suresh K. R.; Murad M. H. et al. Global vascular guidelines on the management of chronic limb-threatening ischemia. J Vasc Surg. 2019, 69, 3S-125S.
3. Dormandy, J.; Belcher, G.; Broos, P.; Eikelboom, B.; Laszlo, G.; Konrad, P.; Moggi, L.; Mueller, U. Prospective study of 713 below-knee amputations for ischaemia and the effect of a prostacyclin analogue on healing. Brit. J Surg. 1994, 81, 33-37.
4. Varu, V. N.; Hogg, M. E.; Kibbe, M. R. Critical limb ischemia. J. Vasc. Surg. 2010, 51, 230-241.
5 Almasri, J.; Adusumalli, J.; Asi, N.; Lakis, S.; Alsawas, M.; Prokop, L. J.; Bradbury, A.; Kolh, P.; Conte, M. S.; Murad, M. H. A systematic review and meta-analysis of revascularization outcomes of infrainguinal chronic limb-threatening ischemia. J. Vasc. Surg. 2019, 69, 126S-136S.
6. Cooke, J. P.; Wilson, A. M. Biomarkers of peripheral arterial disease. J Am. Coll. Cardiol. 2010, 55, 2017-2023.
7. Hyun, S.; Forbang, N. I.; Allison, M. A.; Denenberg, J. O.; Criqui, M. H.; Ix, J. H. Ankle-brachial index, toe-brachial index, and cardiovascular mortality in persons with and without diabetes mellitus. J Vasc. Surg. 2014, 60, 390-395.
8. American Diabetes Association. Peripheral arterial disease in people with diabetes. Diabetes Care 2003, 26, 3333-3341.
9. Screening for peripheral artery disease and cardiovascular disease risk assessment with the ankle-brachial index: US Preventive Services Task Force Recommendation Statement. JAMA 2018, 320, 177-183.
10. McGarrah, R. W.; Crown, S. B.; Zhang, G.-F.; Shah, S. H.; Newgard, C. B. Cardiovascular metabolomics. Circ. Res. 2018, 122, 1238-1258.
11. Wang, Z.; Klipfell, E.; Bennett, B. J.; Koeth, R.; Levison, B. S.; DuGar, B.; Feldstein, A. E.; Britt, E. B.; Fu, X.; Chung, Y.-M.; et al. Gut flora metabolism of phosphatidylcholine promotes cardiovascular disease. Nature 2011, 472, 57-63.
12. Ruiz-Canela, M.; Toledo, E.; Clish, C. B.; Hruby, A.; Liang, L.; Salas-Salvado, J.; Razquin, C.; Corella, D.; Estruch, R.; Ros, E.; et al. Plasma branched-chain amino acids and incident cardiovascular disease in the PREDIMED Trial. Clin. Chem. 2016, 62, 582-592.
13. Shah, S. H.; Sun, J.-L.; Stevens, R. D.; Bain, J. R.; Muehlbauer, M. J.; Pieper, K. S.; Haynes, C.; Hauser, E. R.; Kraus, W. E.; Granger, C. B.; et al. Baseline metabolomic profiles predict cardiovascular events in patients at risk for coronary artery disease. Am. Heart J. 2012, 163, 844-850.
14. Huang, C.-C.; McDermott, M. M.; Liu, K.; Kuo, C.-H.; Wang, S.-Y.; Tao, H.; Tseng, Y. J. Plasma metabolomic profiles predict near-term death among individuals with lower extremity peripheral arterial disease. J. Vasc. Surg. 2013, 58, 989-996.
15. Zagura, M.; Kals, J.; Kilk, K.; Serg, M.; Kampus, P.; Eha, J.; Soomets, U.; Zilmer, M. Metabolomic signature of arterial stiffness in male patients with peripheral arterial disease. Hypertens. Res. 2015, 38, 840-846.
16. Ismaeel, A.; Franco, M. E.; Lavado, R.; Papoutsi, E.; Casale, G. P.; Fuglestad, M.; Mietus, C. J.; Haynatzki, G. R.; Smith, R. S.; Bohannon, W. T.; et al. Altered metabolomic profile in patients with peripheral artery disease. J. Clin. Med. 2019, 8, 1463.
17. Hernandez-Aguilera, A.; Fernandez-Arroyo, S.; Cabre, N.; Luciano-Mateo, F.; Baiges-Gaya, G.; Fibla, M.; Martin-Paredero, V.; Menendez, J. A.; Camps, J.; Joven, J. Plasma energy-balance metabolites discriminate asymptomatic patients with peripheral artery disease. Mediators of Inflammation 2018, 2018, 1-12.
18. Rutherford, R. B.; Baker, J. D.; Ernst, C.; Johnston, K. W.; Porter, J. M.; Ahn, S.; Jones, D. N. Recommended standards for reports dealing with lower extremity ischemia: revised version. J. Vasc. Surg. 1997, 26, 517-538.
19. Al-Khatib, S. M.; Stevenson, W. G.; Ackerman, M. J.; Bryant, W. J.; Callans, D. J.; Curtis, A. B.; Deal, B. J.; Dickfeld, T.; Field, M. E.; Fonarow, G. C.; et al. 2017 AHA/ACC/HRS Guideline for management of patients with ventricular arrhythmias and the prevention of sudden cardiac death: A Report of the American College of Cardiology/American Heart Association Task Force on Clinical Practice Guidelines and the Heart Rhythm Society. J. Am. Coll. Cardiol. 2018, 72, e91-e220.
20. Ou, M.; Song, Y.; Li, S.; Liu, G.; Jia, J.; Zhang, M.; Zhang, H.; Yu, C. LC-MS/MS Method for serum creatinine: comparison with enzymatic method and Jaffe method. PLoS ONE 2015, 10, e0133912.
21. Wild J., Shanmuganathan M., Hayashi M., Potter M., Britz-McKibbin P. Metabolomics for improved treatment monitoring of phenylketonuria: urinary biomarkers for non-invasive assessment of dietary adherence and nutritional deficiencies. Analyst 2019, 144, 6595-6608.
22. Saoi, M.; Li, A.; McGlory, C.; Stokes, T.; von Allmen, M. T.; Phillips, S. M.; Britz-McKibbin, P. Metabolic perturbations from step reduction in older persons at risk for sarcopenia: plasma biomarkers of abrupt changes in physical activity. Metabolites 2019, 9, 134.
23. Azab, S.; Ly, R.; Britz-McKibbin, P. Robust method for high-throughput screening of fatty acids by multisegment injection-nonaqueous capillary electrophoresis-mass spectrometry with stringent quality control. Anal. Chem. 2019, 91, 2329-2336
24. Azab, S. M.; de Souza, R. J.; Teo, K. K.; Anand, S. S.; Williams, N. C.; Holzschuher, J.; McGlory, C.; Phillips, S. M.; Britz-McKibbin, P. Serum non-esterified fatty acids have utility as dietary biomarkers of fat intake from fish, fish oil and dairy in women. J. Lipid Res. 2020, jlr.D120000630, doi:10.1194/jlr.D120000630.
25. Matyash, V.; Liebisch, G.; Kurzchalia, T. V.; Shevchenko, A.; Schwudke, D. Lipid extraction by methyl-tert-butyl ether for high-throughput lipidomics. J. Lipid Res. 2008, 49, 1137-1146.
26. Kuehnbaum, N. L.; Kormendi, A.; Britz-McKibbin, P. Multisegment injection-capillary electrophoresis-mass spectrometry: A high-throughput platform for metabolomics with high data fidelity. Anal. Chem. 2013, 85, 10664-10669.
27. Nori de Macedo, A.; Mathiaparanam, S.; Brick, L.; Keenan, K.; Gonska, T.; Pedder, L.; Hill, S.; Britz-McKibbin, P. The sweat metabolome of screen-positive cystic fibrosis infants: Revealing mechanisms beyond impaired chloride transport. ACS Cent. Sci. 2017, 3, 904-913.
28. Wellington, N.; Shanmuganathan, M.; Souza, R. J. de; Zulyniak, M. A.; Azab, S.; Bloomfield, J.; Mell, A.; Ly, R.; Desai, D.; Anand, S. S.; et al. Metabolic trajectories following contrasting Prudent and Western diets from food provisions: Robust biomarkers of short-term changes in habitual diet. Nutrients 2019, 11, 2407.
29. Yamamoto, M.; Ly, R.; Gill, B.; Zhu, Y.; Moran-Mirabal, J.; Britz-McKibbin, P. Robust and high-throughput method for anionic metabolite profiling: preventing polyimide aminolysis and capillary breakages under alkaline conditions in capillary electrophoresis-mass spectrometry. Anal. Chem. 2016, 88, 10710-10719.
30. DiBattista, A.; Rampersaud, D.; Lee, H.; Kim, M.; Britz-McKibbin, P. High throughput screening method for systematic surveillance of drugs of abuse by multisegment injection-capillary electrophoresis-mass spectrometry. Anal. Chem. 2017, 89, 11853-11861.
31. Dunn, W. B.; Erban, A.; Weber, R. J. M.; Creek, D. J.; Brown, M.; Breitling, R.; Hankemeier, T.; Goodacre, R.; Neumann, S.; Kopka, J.; et al. Mass appeal: metabolite identification in mass spectrometry-focused untargeted metabolomics. Metabolomics 2013, 9, 44-66.
32. Chong, J.; Soufan, 0.; Li, C.; Caraus, I.; Li, S.; Bourque, G.; Wishart, D. S.; Xia, J. MetaboAnalyst 4.0: towards more transparent and integrative metabolomics analysis. Nucleic Acids Research 2018, 46, W486-W494.
33. Yamamoto, M.; Pinto-Sanchez, M. I.; Bercik, P.; Britz-McKibbin, P. Metabolomics reveals elevated urinary excretion of collagen degradation and epithelial cell turnover products in irritable bowel syndrome patients. Metabolomics 2019, 15, 82.
34. Mahieu N. G.; Patti G. J. Systems-level annotation of a metabolomics data set reduces 25 000 features to fewer than 1000 unique metabolites. Anal Chem. 2017; 89: 10397-10406.
35. Gilstrap, L. G.; Wang, T. J. Biomarkers and cardiovascular risk assessment for primary prevention: An update. Clin. Chem. 2012, 58, 72-82.
36. Saoi, M.; Percival, M.; Nemr, C.; Li, A.; Gibala, M.; Britz-McKibbin, P. Characterization of the human skeletal muscle metabolome for elucidating the mechanisms of bicarbonate ingestion on strenuous interval exercise. Anal. Chem. 2019, 91, 4709-4718.
37. Wade, A. M.; Tucker, H. N. Antioxidant characteristics of L-histidine. J. Nutr. Biochem. 1998, 9, 308-315.
38. Tome, D.; Bos, C. Lysine requirement through the human life cycle. J. Nutr. 2007, 137, 1642S-1645S.
39. Castellano, I.; Merlino, A. Gamma-glutamyl transpeptidases: structure and function. In gamma-glutamyl transpeptidases: structure and function; Castellano, I., Merlino, A., Eds.; Springer Briefs in Biochemistry and Molecular Biology; Springer: Basel, 2013; pp. 1-57 ISBN 978-3-0348-0682-4.
40. Steven, S.; Daiber, A.; Dopheide, J. F.; MUnzel, T.; Espinola-Klein, C. Peripheral artery disease, redox signaling, oxidative stress—Basic and clinical aspects. Redox Biol. 2017, 12, 787-797.
41. Saoi, M.; Sasaki, K.; Sagawa, H.; Abe, K.; Kogiso, T.; Tokushige, K.; Hashimoto, E.; Ohashi, Y.; Britz-McKibbin, P. High throughput screening of serum y-glutamyl dipeptides for risk assessment of nonalcoholic steatohepatitis with impaired glutathione salvage pathway. J. Proteome Res. 2020, acs.jproteome.9b00405, doi: 10.1021/acs. jproteome.9b00405.
42. Hajjari, J.; Tahhan, A S; Alkhoder, A.; Kaseer, B.; Naser, A.; Almuwaqqat, Z.; Gafeer, M. M.; Choudhary, F.; Patel, K.; Patel, A. et al. Markers of oxidative stress and peripheral artery disease. J. Am. Coll. Cardiol. 2018, 71, A2076
43. Poesen, R.; Claes, K.; Evenepoel, P.; Loor, H. de; Augustijns, P.; Kuypers, D.; Meijers, B. Microbiota-derived phenylacetylglutamine associates with overall mortality and cardiovascular disease in patients with CKD. J. Am. Soc. Nephrol. 2016, 27, 3479-3487.
44. Sanders Thomas A. B. Omega-6 fatty acids and cardiovascular disease. Circulation 2019, 139, 2437-2439.
45. Matti, M.; Wu Jason H. Y.; Imamura Fumiaki; Del Gobbo Liana C.; Fretts Amanda; de Goede Janette; Shi Peilin; Tintle Nathan; Wennberg Maria; Aslibekyan Stella; et al. Biomarkers of dietary omega-6 fatty acids and incident cardiovascular disease and mortality. Circulation 2019, 139, 2422-2436.
46. Grenon S. M.; Hughes-Fulford M.; Rapp J.; Conte M. S. Polyunsaturated fatty acids and peripheral artery disease. Vasc. Med. 2012, 17, 51-63.
47. Ruiz-Canela, M.; Estruch, R.; Corella, D.; Salas-Salvado, J.; Martinez-Gonzalez, M. A. Association of Mediterranean diet with peripheral artery disease: The PREDIMED Randomized Trial. JAMA 2014, 311, 415. 22
48. Weitkunat, K.; Schumann, S.; Nickel, D.; Hornemann, S; Petzke, K. J.; Schulze, M. B.; Pfeiffer, A. F.; Klaus, S. Odd-chain fatty acids as a biomarker for dietary fiber intake: a novel pathway for endogenous production from propionate. Am. Clin. Nutr. 2017, 105, 1544-1551.
49. Senyilmaz-Tiebe D.; Pfaff D. H.; Virtue S.; Schwarz K. V.; Fleming T.; Altamura S.; Muckenthaler, M. U.; Okun J. G.; Vidal-Puig, A.; Nawroth P.; Teleman, A. A. Dietary stearic acid regulates mitochondria in vivo in humans. Nat. Commun. 2018, 9, 3129.
50. Weiss, D. J.; Casale, G. P.; Koutakis, P.; Nella, A. A.; Swanson, S. A.; Zhu, Z.; Miserlis, D.; Johanning, J. M.; Pipinos, I. I. Oxidative damage and myofiber degeneration in the gastrocnemius of patients with peripheral arterial disease. J. Transl. Med. 2013, 11, 230.
51. Senthong, V.; Wang, Z.; Fan, Y.; Wu, Y.; Hazen, S. L.; Tang, W. H. W. Trimethylamine N-oxide and mortality risk in patients with peripheral artery disease. J. Am. Heart Assoc. 2016, 5, e004237.
52. Roncal, C.; Martinez-Aguilar, E.; Orbe, J.; Ravassa, S.; Fernandez-Montero, A.; Saenz-Pipaon, G.; Ugarte, A.; Estella-Hermoso de Mendoza, A.; Rodriguez, J. A.; Fernandez-Alonso, S.; et al. Trimethylamine-n-oxide (TMAO) predicts cardiovascular mortality in peripheral artery disease. Sci. Rep. 2019, 9, 15580.
53. Mlekusch W.; Exner M.; Sabeti S.; Amighi J.; Schlager 0.; Wagner, 0.; Minar, E.; Schillinger M. Serum creatinine predicts mortality in patients with peripheral artery disease: influence of diabetes and hypertension. Atherosclerosis 2004, 175, 361-367.
54. Ormstad, H.; Verkerk, R.; Sandvik, L. Serum phenylalanine, tyrosine, and their ratio in acute ischemic stroke: on the trail of a biomarker? J. Mol. Neurosci. 2016, 58, 102-108.

1. A method of diagnosing peripheral artery disease (PAD) in a mammalian subject comprising:
i) detecting in a biological sample from the subject the level of one or more metabolic biomarkers selected from the group consisting of: creatine, creatinine, carnitine, propionylcarnitine, cystine, lysine, tyrosine, histidine, phenylacetylglutamine, oxoproline, arginine, monomethylarginine, a fatty acid biomarker selected from the group consisting of stearic acid, linoleic acid, heptadecanoic acid, palmitic acid, oleic acid, heptadecenoic acid, pentadecanoic acid and eicosadienoic acid, and a ratiometric biomarker comprising at least one of the metabolic biomarkers; comparing the level of the one or more detected biomarkers to the corresponding level in a non-PAD control; and
iii) determining that the subject has PAD when the level of the one or more detected biomarkers is statistically different from the corresponding level in a non-PAD control.

2. The method of claim 1, wherein the level of at least two biomarkers is detected in the sample.

3. The method of claim 2, wherein only one of the biomarkers is a fatty acid biomarker.

4. The method of claim 1, wherein one biomarker is a ratiometric biomarker.

5. The method of claim 4, wherein the ratiometric biomarker comprises at least one of stearic acid, linoleic acid, heptanoic acid, creatinine, carnitine and cystine.

6. The method of claim 4, wherein the ratiometric biomarker is selected from stearic acid:carnitine and arginine:propionylcarnitine.

7. The method of claim 1, comprising the additional step of treating the subject with one or more treatments selected from the group consisting of: medication to treat leg pain, a cholesterol-lowering medication, medication to treat high blood pressure, medication to prevent blood clots angioplasty, thrombolytic therapy, surgery, exercise therapy and modified diet.

8. The method of claim 1, wherein the biological sample is a blood sample.

9. The method of claim 1, wherein the subject is a human

10. A method of distinguishing chronic limb-threatening ischemia (CLTI) from intermittent claudication (IC) in a mammalian subject having PAD comprising:
i) detecting in a biological sample from the subject the level of one or more metabolic biomarkers selected from the group consisting of: creatine, creatinine, carnitine, propionylcarnitine, cystine, lysine, tyrosine, histidine, phenylacetylglutamine, oxoproline, arginine, monomethylarginine, a fatty acid biomarker selected from the group consisting of stearic acid, linoleic acid, heptadecanoic acid, palmitic acid, oleic acid, heptadecenoic acid, pentadecanoic acid and eicosadienoic acid, and a ratiometric biomarker comprising at least one of the metabolic biomarkers;
ii) comparing the level of the one or more detected biomarkers to the corresponding level in an IC control; and
iii) determining that the subject has CLTI when the level of the one or more detected biomarkers is statistically different from the corresponding level in the IC control.

11. The method of claim 10, wherein the level of at least two biomarkers is detected in the sample.

12. The method of claim 11, wherein only one of the biomarkers is a fatty acid biomarker.

13. The method of claim 10, wherein one of the biomarkers is a ratiometric biomarker.

14. The method of claim 13, wherein the ratiometric biomarker comprises at least one of stearic acid, linoleic acid, heptanoic acid, creatinine, carnitine and cystine.

15. The method of claim 13, wherein the ratiometric biomarker is selected from stearic acid:carnitine and arginine:propionylcarnitine.

16. The method of claim 10, comprising the additional step of treating the subject with one or more treatments selected from the group consisting of: medication to treat leg pain, a cholesterol-lowering medication, medication to treat high blood pressure, medication to prevent blood clots angioplasty, thrombolytic therapy, surgery, exercise therapy and modified diet.

17. The method of claim 1, wherein the biological sample is treated to remove proteins prior to biomarker detection.

18. The method of claim 1, wherein the biological sample is acid hydrolyzed and prior to biomarker detection.
